(19) United States
(12) Patent Application Publication
(10) Pub. No.: US 2014/0330121 A1
(43) Pub. Date: Nov. 6, 2014

(54) ULTRASONIC IMAGING APPARATUS AND CONTROL METHOD THEREOF

(71) Applicant: SAMSUNG ELECTRONICS CO., LTD., Suwon-si (KR)

(72) Inventor: Yun Tae KIM, Hwaseong-si (KR)

(73) Assignee: SAMSUNG ELECTRONICS CO., LTD., Suwon-si (KR)

(21) Appl. No.: 14/186,050

(22) Filed: Feb. 21, 2014

(30) Foreign Application Priority Data
May 6, 2013 (KR) 10-2013-0050900

(51) Int. Cl. A61B 8/08

(52) U.S. Cl. CPC A61B 8/485; A61B 8/483
USPC 600/438

(57) ABSTRACT

An ultrasonic imaging apparatus includes an ultrasonic probe, a volume data generator to generate a plurality of volume data corresponding to echo signals received as the ultrasonic probe transmits the ultrasonic signals to the object a plurality of times before and while external stress is applied to the object, an elasticity data generator to generate elasticity data based on displacement of the plurality of volume data, a controller to adjust parameters of volume rendering using the elasticity data, and an image processor to perform the volume rendering using the adjusted parameters and generate a volume-rendered image.

Accordingly, a multi-dimensional ultrasonic image of a target region of an object to be diagnosed in which lesion areas are separated from non-lesion tissues may be output. Thus, the information regarding the surface of the target region and the inside volume of the target region may be acquired.

### CROSS-REFERENCE TO RELATED APPLICATION

This application claims priority from Korean Patent Application No. 10-2013-0050900, filed on May 6, 2013 in the Korean Intellectual Property Office, the disclosure of which is incorporated herein by reference in its entirety.

### BACKGROUND

1. Field

Apparatuses and methods consistent with exemplary embodiments relate to an ultrasonic imaging apparatus that outputs a multi-dimensional ultrasonic image using elasticity data of an object and a control method thereof.

2. Description of the Related Art

An ultrasonic imaging apparatus radiates ultrasonic waves toward a target region of an object to be diagnosed from the surface of the object and detects reflected signals from the target region, i.e., ultrasonic echo signals, to generate an image of the target region such as a soft tissue tomogram or a blood stream tomogram, thereby providing information regarding the target region.

The ultrasonic imaging apparatus is small and inexpensive, as compared to other image diagnostic apparatuses, such as an X-ray diagnostic apparatus, a computed tomography (CT) scanner, a magnetic resonance imaging (MRI) apparatus, and a nuclear medicine diagnostic apparatus, and is thus widely used for heart diagnosis, celiac diagnosis, urinary diagnosis, as well as obstetric diagnosis due to non-invasive and nondestructive characteristics thereof.

In particular, a three-dimensional (3D) ultrasonic imaging apparatus generates a 3D ultrasonic image of an object by acquiring 3D data regarding the object using a probe, or the like, and performing volume rendering of the acquired 3D data, and then visualizes the 3D ultrasonic image on a display device. In this case, when a target region is a fetus, information regarding the surface, such as the eyes, nose, and mouse, should be visualized. However, when the target region is an internal organ such as the thyroid, kidney, and liver, information regarding the inside of the organ, i.e., information regarding lesion areas, should be obtained instead of information regarding the surface of the organ.

### SUMMARY

Exemplary embodiments may address at least the above problems and/or disadvantages and other disadvantages not described above. Also, the exemplary embodiments are not required to overcome the disadvantages described above, and an exemplary embodiment may not overcome any of the problems described above.

One or more exemplary embodiments provide an ultrasonic imaging apparatus to output a multi-dimensional ultrasonic image of a target region of an object to be diagnosed in which lesion areas are separated from non-lesion tissues using elasticity data of the object and a method of controlling the ultrasonic imaging apparatus.

In accordance with an aspect of an exemplary embodiment, an ultrasonic imaging apparatus and a method of controlling the ultrasonic imaging apparatus are provided.

The ultrasonic imaging apparatus includes an ultrasonic probe to transmit ultrasonic signals to an object and receive echo signals reflected from the object, a volume data generator to generate a plurality of volume data corresponding to a plurality of echo signals received as the ultrasonic probe transmits the ultrasonic signals to the object plural times before or while external stress is applied to the object, an elasticity data generator to generate elasticity data based on displacement of the plurality of volume data, a controller to adjust parameters of volume rendering using the elasticity data, and an image processor to perform the volume rendering using the adjusted parameters and generate a volume-rendered image.

The parameters adjusted by the controller may include at least one of an opacity value of a voxel and a voxel value.

The opacity value may be established as a one-dimensional increasing function with respect to the elasticity value and adjusted proportionally to the elasticity value.

The opacity value may be established as a two-dimensional increasing function with respect to the elasticity value and the voxel value and adjusted proportionally to the elasticity value and the voxel value, or the opacity value may be established as a two-dimensional increasing function with respect to the elasticity value and a gradient value and adjusted proportionally to the elasticity value and the gradient value.

The opacity value may be established as a two-dimensional increasing function with respect to the elasticity value and the voxel value while only increasing proportionally to the voxel value and adjusted to be established as a one-dimensional increasing function with respect to the voxel value when the elasticity value is 0.

The opacity value may be established as a two-dimensional increasing function with respect to the elasticity value and the voxel value while only increasing proportionally to the voxel value and adjusted to be established as a one-dimensional increasing function with respect to the elasticity value when the voxel value is 0.

The voxel value may be established as a one-dimensional increasing function with respect to the elasticity value and adjusted proportionally to the elasticity value.

The voxel value is adjusted by Equation 1 below:

Voxelₒᵤₜ=Voxelᵢₙ×ƒ(e) Equation 1

In Equation 1, e is an elasticity value, f is a value from 0 to 1, the function of the voxel value is a one-dimensional increasing function dependent upon the elasticity value, Voxelᵢₙ is a voxel value before adjustment, and Voxelₒᵤₜ is a voxel value after adjustment.

The parameters adjusted by the controller may further include a color value of the voxel.

The color value may be adjusted using the opacity value of the voxel and the voxel value.

The ultrasonic imaging apparatus may further include a volume data adjuster to align geometrical positions of the plurality of volume data generated by the volume data generator and geometrical positions of the elasticity data generated by the elasticity data generator.

In accordance with an aspect of an exemplary embodiment, a method of controlling an ultrasonic imaging apparatus may include receiving a plurality of echo signals as a probe transmits ultrasonic signals to an object plural times before and while external stress is applied to the object, generating a plurality of volume data corresponding to the plurality of echo signals, generating elasticity data based on displacement of the plurality of volume data, adjusting parameters of volume rendering using the elasticity data, and performing volume rendering using the adjusted parameters and generating a volume rendered image.

### BRIEF DESCRIPTION OF THE DRAWINGS

The above and/or other aspects will become more apparent by describing certain exemplary embodiments, with reference to the accompanying drawings, in which:

FIG. 1 is a perspective view illustrating an outer appearance of an ultrasonic imaging apparatus according to an exemplary embodiment;

FIG. 2 is a block diagram of an ultrasonic imaging apparatus according to an exemplary embodiment;

FIG. 3 is a diagram illustrating a plurality of two-dimensional (2D) cross-sectional images;

FIG. 4 is a diagram exemplarily illustrating volume data;

FIG. 5 is a diagram for describing a process of generating elasticity data;

FIG. 6 is a block diagram illustrating a controller of an ultrasonic imaging apparatus according to an exemplary embodiment;

FIG. 7 is a diagram for describing a method of adjusting geometrical positions of volume data;

FIG. 8 is a diagram for describing a three-dimensional (3D) scan conversion of volume data;

FIGS. 9A and 9B illustrate graphs of one-dimensional (1D) opacity transfer functions using elasticity values according to an exemplary embodiment;

FIGS. 10A, 10B, 10C, and 10D illustrate graphs of 2D opacity transfer functions using elasticity values according to an exemplary embodiment;

FIGS. 11A and 11B illustrate graphs of 2D opacity transfer functions using elasticity values according to an exemplary embodiment;

FIGS. 12A and 12B illustrate graphs of voxel value adjustment functions according to an exemplary embodiment;

FIG. 13 is a diagram for describing volume ray casting;

FIG. 14 is a diagram illustrating a 3D ultrasonic image acquisition by an ultrasonic imaging apparatus; and

FIG. 15 is a flowchart illustrating a method of controlling an ultrasonic imaging apparatus according to an exemplary embodiment.

### DETAILED DESCRIPTION

Certain exemplary embodiments are described in greater detail below with reference to the accompanying drawings.

In the following description, the same drawing reference numerals are used for the same elements even in different drawings. The matters defined in the description, such as detailed construction and elements, are provided to assist in a comprehensive understanding of exemplary embodiments. Thus, it is apparent that exemplary embodiments can be carried out without those specifically defined matters. Also, well-known functions or constructions are not described in detail since they would obscure exemplary embodiments with unnecessary detail.

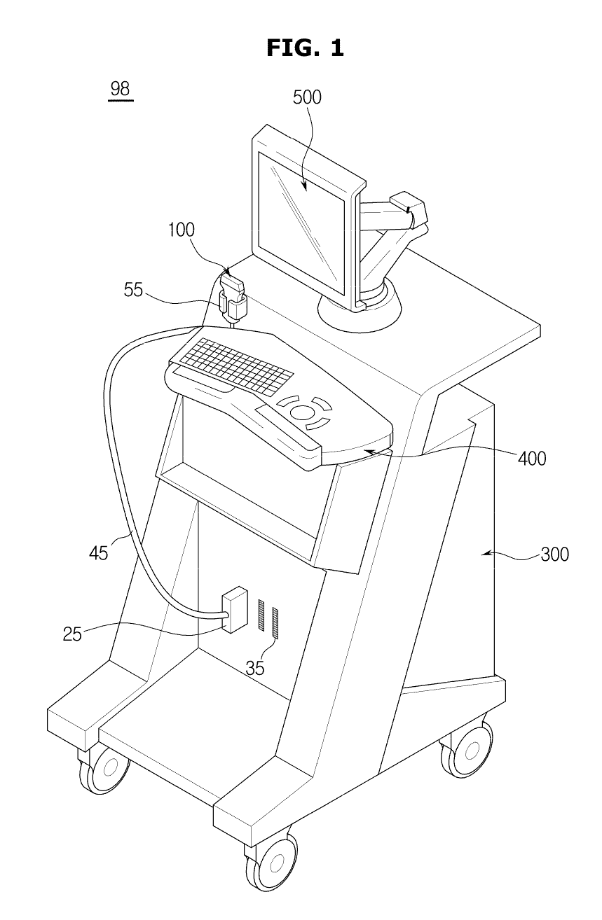

FIG. 1 is a perspective view illustrating an outer appearance of an ultrasonic imaging apparatus according to an exemplary embodiment.

As illustrated in FIG. 1, an ultrasonic imaging apparatus 98 includes a probe 100, a main body 300, an input unit 400, and a display 500.

The probe 100 that directly contacts an object may transmit and receive ultrasonic signals in order to acquire an ultrasonic image of a target region of the object to be diagnosed. Here, the object may be a living body of human or animals, and the target region may be a tissue in the living body such as, the liver, a blood vessel, a bone, a muscle, and the like.

One end of a cable 45 is connected to the probe 100, and the other end of the cable 45 may be connected to a male connector 25. The male connector 25 connected to the other end of the cable 45 may be physically coupled to a female connector 35.

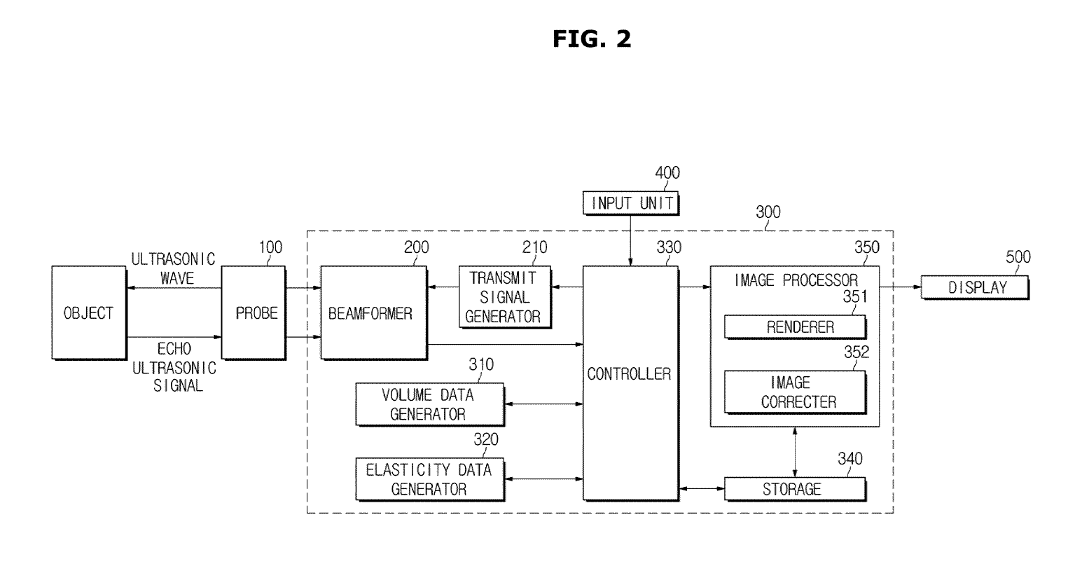

The main body 300 may accommodate major constituent elements of the ultrasonic imaging apparatus, for example, a transmit signal generator 210 of FIG. 2. When an operator inputs a command to perform ultrasonic diagnosis, the transmit signal generator 210 may generate a transmit signal and transmit the transmit signal to the probe 100.

The main body 300 may have at least one female connector 35. The female connector 35 may be physically coupled to the male connector 25 connected to the cable 45 such that the main body 300 and the probe 100 may transmit and receive signals generated thereby. For example, the transmit signal generated by the transmit signal generator 210 may be transmitted to the probe 100 via the male connector 25 connected to the female connector 35 of the main body 300 and the cable 45.

In addition, although not illustrated in FIG. 1, a plurality of casters, capable of fixing the ultrasonic imaging apparatus to a predetermined position or moving the ultrasonic imaging apparatus in a predetermined direction, may be installed at lower portions of the main body 300.

The input unit 400 receives a command regarding operation of the ultrasonic imaging apparatus. For example, the input unit 400 may receive a command to initiate ultrasonic diagnosis, a command as to whether a parameter of a volume rendering to be adjusted using an elasticity value is an opacity value or a voxel value, or a command as to whether information to be detected is information regarding the surface or information regarding the inside of the target region. The command input by the input unit 400 may be transmitted to the main body 300 via a wired or wireless communication network.

The input unit 400 may include at least one of a switch, a keyboard, a trackball, and a touchscreen, but is not limited thereto.

The input unit 400 may be disposed at an upper portion of the main body 300 as illustrated in FIG. 1. However, a foot switch, a foot pedal, and the like may also be disposed at lower portions of the main body 300.

At least one probe holder 55 to hold the probe 100 may be mounted around the input unit 400. Thus, the operator may store the probe in the probe holder 55 when the ultrasonic imaging apparatus is not in use.

The display 500 may display an ultrasonic image acquired during the ultrasonic diagnosis on a screen. The display 500 may be coupled to the main body 300, and may also be implemented detachably from the main body.

The display 500 may be a cathode ray tube (CRT), a liquid crystal display (LCD), an organic light emitting diode (OLED) display, or the like, but is not limited thereto.

Although not illustrated in FIG. 1, the display 500 may include a separate sub-display that displays applications regarding operation of the ultrasonic imaging apparatus, such as a menu or guidelines required for ultrasonic diagnosis.

Hereinafter, the ultrasonic imaging apparatus will be described in more detail with reference to FIGS. 2 to 14.

FIG. 2 is a block diagram of an ultrasonic imaging apparatus according to an exemplary embodiment.

The probe 100 includes a plurality of transducer elements and converts an electrical signal into an ultrasonic signal, and vice versa. The probe 100 transmits ultrasonic signals to an object and receives echo signals reflected from the object.

Particularly, when the probe receives current from an external power supply device or an internal power storage device, such as a battery, the plurality of transducer elements vibrate to generate ultrasonic signals and radiate the generated ultrasonic signals toward an external object. The transducer elements receive echo signals reflected from the object and vibrate in response to the received echo signals, thereby generating current having frequencies corresponding to the vibration frequencies thereof.

Referring to FIG. 2, the main body 300 may include a transmit signal generator 210, a beamformer 200, a volume data generator 310, an elasticity data generator 320, a controller 330, a storage 340, and an image processor 350.

The transmit signal generator 210 may generate a transmit signal in accordance with a control command from the controller 330 and transmit the generated transmit signal to the probe 100. Here, the transmit signal is a high-pressure electrical signal to vibrate the transducer elements of the probe 100.

Since the beamformer 200 converts an analog signal into a digital signal, and vice versa, the beamformer 210 aids communications between the probe 100 and the main body 300 by converting the transmit signal (digital signal) generated by the transmit signal generator 210 into an analog signal or by converting the echo signal (analog signal) received from the probe 100 into a digital signal.

The beamformer 200 may apply a time delay to the digital signal in consideration of position and focus point of each of the transducer elements in order to remove a time difference of arrival at a focus point between the ultrasonic waves or a time difference of arrival at each transducer element from the focus point between the ultrasonic echo signals.

A process of concentrating ultrasonic waves, which are simultaneously emitted by a plurality of transducer elements, into a focus point is referred to as focusing. The beamformer 210 performs transmit focusing, by which ultrasonic waves respectively generated by the transducer elements are sequentially emitted in a predetermined order to remove time difference of arrival at the focus point between the ultrasonic waves, and receive focusing, by which the ultrasonic echo signals are simultaneously aligned using predetermined time difference to remove time difference of arrival at each transducer element between the ultrasonic echo signals.

The beamformer 210 may be disposed in the main body as illustrated in FIG. 2 or may be disposed in the probe 100 performing functions thereof.

The volume data generator 310 may generate a plurality of volume data before or while external stress is applied to the object corresponding to a plurality of echo signals received as the probe 100 transmits a plurality of ultrasonic signals. Here, the echo signal is a signal having undergone a variety of processes by a signal processor 332, which will be described later.

For example, an echo signal reflected from the ultrasonic signal transmitted from the probe 100 toward the object before the external stress is applied to the object is referred to as a first echo signal, and an echo signal reflected from the ultrasonic signal transmitted from the probe 100 toward the object while external stress is applied to the object is referred to as a second echo signal. In this case, the volume data generator 310 may generate first volume data corresponding to the first echo signal and second volume data corresponding to the second echo signal.

In this case, the external stress may be applied by applying stress, in a proceeding direction of the ultrasonic waves, for example, static stress using a hand of the operator or the probe 100, a high-pressure ultrasonic pulse, or a mechanical vibration, or by applying stress, in a direction perpendicular to the proceeding direction of the ultrasonic waves, such as, shearwave using transverse wave. However, the present exemplary embodiment is not limited thereto.

In addition, when the object is three-dimensionally visualized, two-dimensional (2D) cross-sectional images of the object are acquired corresponding to the echo signals received by the probe 100, and the 2D cross-sectional images are sequentially stacked in the corresponding order thereof to generate a set of discrete three-dimensional (3D) alignments. The volume data refers to a set of the 3D alignments.

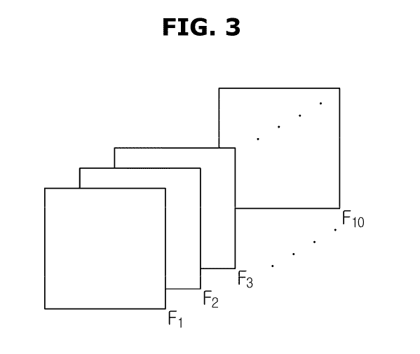

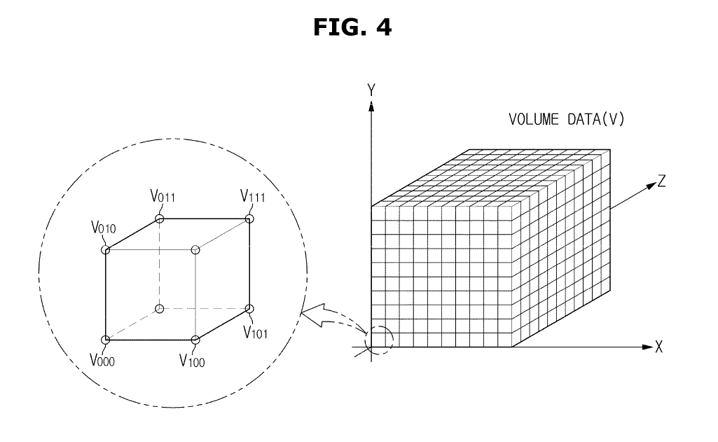

Referring to FIGS. 3 and 4, an example of the volume data will be described. FIG. 3 illustrates a plurality of 2D cross-sectional images. FIG. 4 illustrates volume data.

As illustrated in FIG. 3, a plurality of 2D cross-sectional images F1, F2, F3, . . . , F10 of the object are acquired corresponding to the echo signals received by the probe 100. 3D volume data of the object as illustrated in FIG. 4 may be generated via alignment of the acquired 2D images F1, F2, F3, . . . , F10 in a 3D shape in the corresponding positions thereof and data interpolation of the cross-sectional images.

The volume data may be constituted with a plurality of voxels. The term “voxel” is formed through combination of the terms “volume” and “pixel”. While pixel refers to a single point in a 2D plane, voxel refers to a single point in a 3D space. Thus, a pixel has X- and Y-coordinates, whereas a voxel has X-, Y-, and Z-coordinates.

Accordingly, when the volume data is a group V of voxels, and a spatial 3D coordinate value indicating the location of the voxel is (x, y, z), the voxel may be represented by V_{xyz}.

For example, as illustrated in FIG. 4, a voxel having a spatial coordinate value of (0,0,0) may be represented by V₀₀₀, a voxel having a spatial coordinate value of (1,0,0) may be represented by V₁₀₀, and a voxel having a spatial coordinate value of (0,1,0) may be represented by V₀₁₀.

In addition, a voxel value va corresponding to a voxel V_{xyz} may be represented by V(x,y,z)=va. Here, the voxel value va may be a scalar value or a vector value, and the volume data may be classified according to the type of the voxel.

For example, a voxel value represented by a binary number of 0 or 1 may be referred to as a binary volume data, and a voxel value represented by a measurable value, such as density and temperature, may be referred to as multi-valued volume data. In addition, a voxel value represented by a vector such as speed or RGB color may be referred to as vector volume data.

Optical properties of the voxel, such as opacity values and color values, may be calculated using the voxel values. The opacity value may be calculated using an opacity transfer function that defines the relationship between the voxel values and the opacity values, and the color value may be calculated using a color transfer function that defines the relationship between the voxel values and the color values.

A plurality of volume data or voxel values generated by the volume data generator 310 may be stored in the storage 340.

The elasticity data generator 320 may calculate elasticity values of the voxels based on displacement of the plurality of volume data and generate 3D elasticity data of the object.

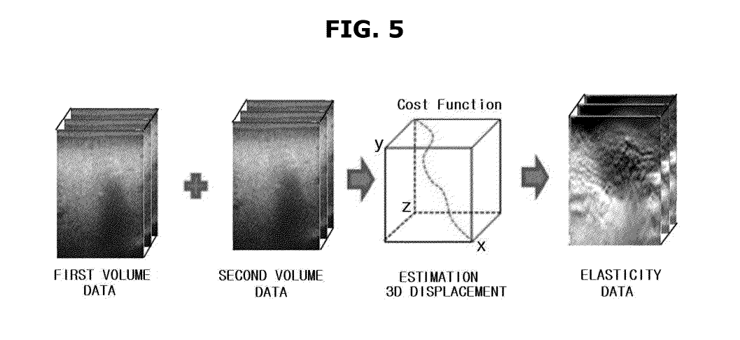

FIG. 5 is a diagram for describing a process of generating elasticity data.

Referring to FIG. 5, the probe 100 transmits ultrasonic signals toward the object before and while external stress is applied. Correspondingly, the volume data generator 310 separately generates first volume data and second volume data. A 3D cross-correlation is calculated using the first volume data and the second volume data on a per voxel basis, thereby generating cost function values. A 3D elasticity data is generated through an optimization algorithm, such as least squares and dynamic programming, to find a minimum cost function value.

That is, displacement of the voxels, namely variation of the voxel values, is calculated by use of the first volume data and the second volume data, which correspond to each other, and then elasticity values of the voxels are calculated from the displacement.

Here, the elasticity value refers to an ability of a material to return to the original shape thereof when external stress is removed and is inverse proportional to a strain rate that refers to the degree of deformation caused by the external stress. Thus, in this case, displacement of the voxel corresponding to the strain rate is proportional to the elasticity value. That is, as hardness of the target region of the object increases, displacement of the voxel decreases, but the elasticity value of the voxel increases.

For example, when the target region contains cancerous or tumor-like lesions, the voxel values of the lesions are not significantly changed by external stress in comparison with that before the external stress is applied thereto. That is, displacement of the voxels decreases in the lesion areas, so that the calculated elasticity values increase. On the other hand, in soft tissues which are non-lesion areas, displacement of the voxels increases by the external stress, so that the calculated elasticity values decrease.

The elasticity data generated by the elasticity data generator 320 form a set of 3D alignments, similarly to the plurality of volume data generated by the volume data generator 310. Here, the voxel values of the voxels constituting the elasticity data indicate elasticity values.

As described above, the generated elasticity data or elasticity values may be stored in the storage 340.

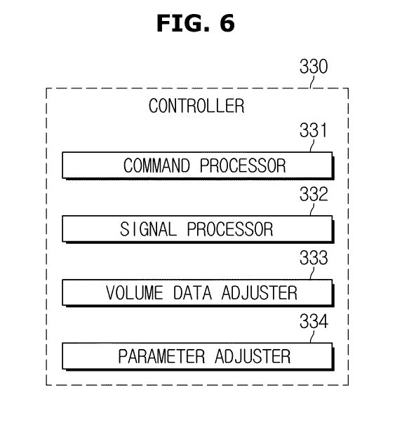

FIG. 6 is a block diagram illustrating a controller 330 of an ultrasonic imaging apparatus according to an exemplary embodiment.

The controller 330 may include a command processor 331, a signal processor 332, a volume data adjuster 333, and a parameter adjuster 334.

The command processor 331 may output a control command signal to the transmit signal generator 210.

When an operator inputs a command to perform ultrasonic diagnosis into the input unit 400, the command processor 331 outputs a command signal to generate a transmit signal to the transmit signal generator 210.

The command processor 331 may output a control command signal to the image processor 350.

The command processor 331 may output a command signal to display an image generated during ultrasonic diagnosis on the display 500 to the image processor 350.

The command processor 331 may simultaneously output a command signal regarding a screen display mode to the image processor 350. In this case, the screen display mode may include an A-mode to display the intensity of the echo signal as amplitude, a B-mode using brightness or luminance, an M-mode to display a distance from a moving target region using variation of time, a D-mode using a pulse wave or continuous wave, and a color flow mapping (CFM)-mode to display a color image using the Doppler effect, but is not limited thereto. The command signal may be output using an automatically selected display mode according to the position, size, and shape of the target region or a display mode input by the operator via the input unit 400.

The signal processor 332 may include an overall gain control process to amplify the overall amplitude of the echo signal since it is difficult to display the echo signal output from the beamformer 200 in a real image due to small amplitude thereof.

Since the ultrasonic waves are attenuated while passing through a medium of the object, the signal processor 332 may perform time gain compensation (TCG) to amplify the echo signal proportionally to the distance from the target region.

The signal processor 332 may conduct filtering, i.e., remove low level noises from the echo signal, to obtain a clear signal.

The volume data adjuster 333 may align geometrical positions of the plurality of volume data generated by the volume data generator 310 in a one-to-one corresponding manner, and align the geometrical positions of the volume data and elasticity data generated by the elasticity data generator 310 in a one-to-one corresponding manner.

First, the volume data adjuster 333 may align the geometrical positions of the plurality of volume data generated by the volume data generator 310 in a one-to-one corresponding manner before generating elasticity data.

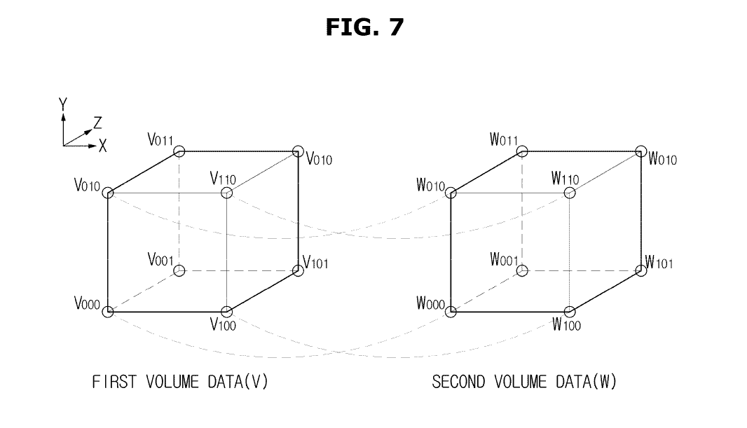

FIG. 7 is a diagram for describing a method of aligning geometrical positions of volume data in a one-to-one corresponding manner.

Referring to FIG. 7, when volume data generated as the probe 100 transmits ultrasonic waves toward the object before and while external stress is applied thereto are respectively referred to as first volume data V and second volume data W, the geometrical positions of the two volume data may be aligned in a one-to-one corresponding manner such that V₀₀₀ corresponds to W₀₀₀, V₁₀₀ corresponds to W₁₀₀, V₀₁₀ corresponds to W₀₁₀, and V₁₁₀ corresponds to W₁₁₀. In the same manner, the geometrical positions of the two volume data may be aligned in a one-to-one corresponding manner such that the voxels V_{xyz} of the first volume data V respectively correspond to the voxels W_{xyz} of the second volume data W.

Then, after generating elastic data using displacement of the volume data, the geometrical positions of which are aligned, the geometrical positions between the volume data and the elasticity data are aligned in a one-to-one corresponding manner.

For example, volume data generated as the probe 100 transmits ultrasonic waves toward the object before and while an external stress is applied thereto are respectively referred to as first volume data V and second volume data W, and elastic data generated based on the displacement of the two volume data is referred to as elastic data E. In this case, the geometrical positions of the volume data may be aligned to the geometrical positions of the elasticity data E, such that V₀₀₀ corresponds to E₀₀₀, V₁₀₀ corresponds to E₁₀₀, V₀₁₀ corresponds to E₀₁₀, and V₁₁₀ corresponds to E₁₁₀. In the same manner, the geometrical positions of the volume data may be aligned to the geometrical positions of the elasticity data E such that the voxels V_{xyz} of the first volume data V correspond to the voxels E_{xyz} of the elasticity data E.

Here, since the geometrical positions of the two volume data, i.e., the first volume data and the second volume data, are adjusted as described above, the geometrical positions of the two volume data are aligned to the geometrical positions of the elasticity data in a one-to-one corresponding manner.

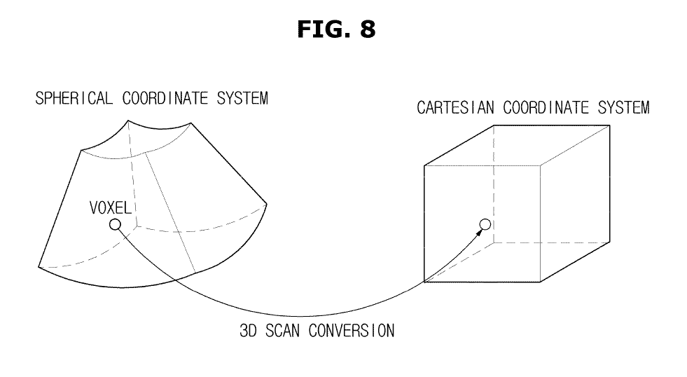

The volume data adjuster 333 may also perform 3D scan conversion of the volume data as illustrated in FIG. 8.

FIG. 8 is a diagram for describing 3D scan conversion of volume data.

Since a display device has a Cartesian coordinate system, the volume data of the object needs to be converted so as to conform to the Cartesian coordinate system to three-dimensionally visualize the volume data on the screen of the display device. That is, when the volume data generated by the volume data generator 310 is defined on a spherical coordinate system as illustrated in FIG. 8 on the left, coordinate conversion is required to visualize the volume data on the screen of the display device. Thus, the volume data adjuster 333 conducts 3D scan conversion to convert the volume data of each voxel defined in the spherical coordinate system of FIG. 8 on the left into the volume data of a corresponding position defined in the Cartesian coordinate system as illustrated in FIG. 8 on the right.

The parameter adjuster 334 may adjust parameters of volume rendering such as a voxel value, an opacity value, and a color value, using electricity data generated by the elasticity data generator 320 before performing the volume rendering. Here, the adjusted voxel value, the opacity value, and the color value are a voxel value, an opacity value, and a color value of each voxel constituting the volume data among volume data generated before external stress is applied to the object.

First, the parameter adjuster 334 may adjust at least one of the opacity value of the voxel and the voxel value.

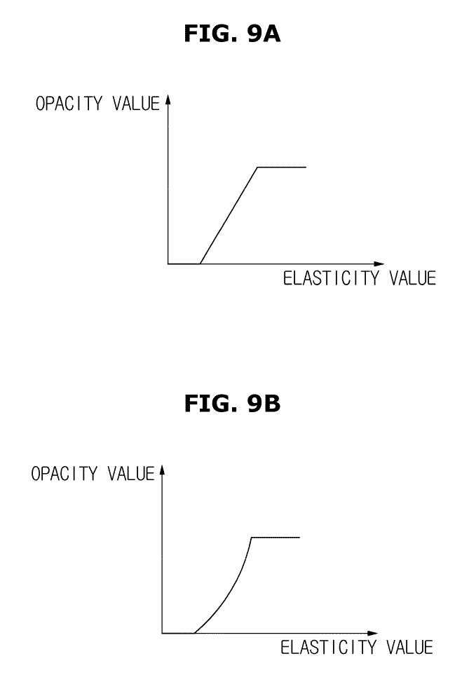

The opacity value is established as a one-dimensional (1D) increasing function with respect to elasticity values and may be adjusted to increase proportionally to the elasticity values. In this regard, the 1D increasing function is referred to as a 1D opacity transfer function, and examples thereof are illustrated in FIGS. 9A and 9B.

FIGS. 9A and 9B illustrate graphs of 1D opacity transfer functions using elasticity values according to an exemplary embodiment.

In the functions illustrated in FIGS. 9A and 9B, an opacity value of a voxel having a low elasticity value is set to 0, and an opacity value of a voxel having a high elasticity value is increased. Thus, when the target region contains cancerous or tumor-like lesions, lesion areas are represented to be opaque due to high elasticity values, and non-lesion areas of soft tissue are represented to be transparent due to low elasticity values according to these functions.

The 1D opacity transfer function may have a linear structure as illustrated in FIG. 9A or a nonlinear structure as illustrated in FIG. 9B.

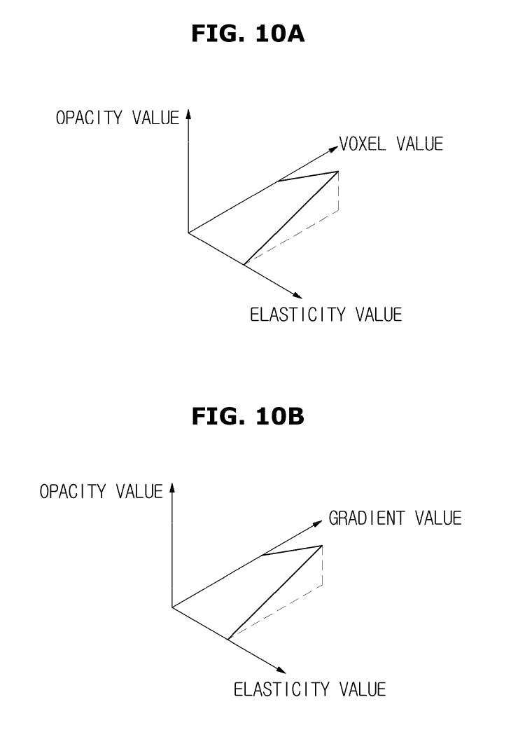

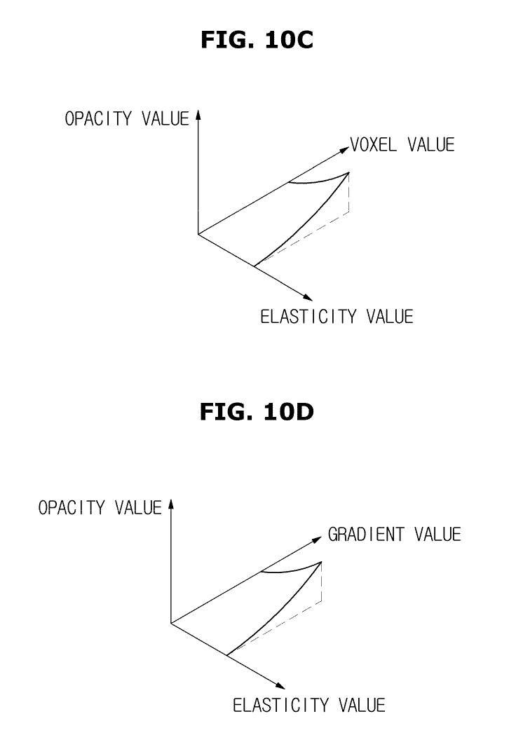

The opacity values may be established as a 2D increasing function with respect to elasticity values and voxel values so as to increase proportionally to the elasticity value and the voxel value. Alternatively, the opaque values may be established as a 2D increasing function with respect to elasticity values and gradient values so as to increase proportionally to the elasticity values and the gradient values. Here, the 2D increasing function is referred to as a 2D opacity transfer function, and examples thereof are illustrated in FIG. 10A to 10D.

FIGS. 10A to 10D illustrate graphs of 2D opacity transfer functions using elasticity values according to an exemplary embodiment.

Referring to FIGS. 10A and 10C, the elasticity value and the voxel value are used as input variables. An opacity value of a voxel having a low elasticity value and a low voxel value is set to 0 to make the voxel transparent, and an opacity value of a voxel having a high elasticity value and a high voxel value is increased to raise the degree of reflection of the opacity value on the image generated as a result of volume rendering. Thus, when the target region contains cancerous or tumor-like lesions, lesion areas are represented to be opaque due to high elasticity values and high voxel values, and non-lesion areas of soft tissue are represented to be transparent due to low elasticity values and low voxel values according to these functions.

FIGS. 10B and 10D are graphs using gradient values instead of the voxel values. That is, the elasticity value and the gradient value are used as input variables. An opacity value of a voxel having a low elasticity value and a low gradient value is set to 0 such that the voxel is represented to be transparent, and an opacity value of a voxel having a high elasticity value and a high gradient value is increased to raise the degree of reflection of the opacity value on the image generated as a result of volume rendering.

Thus, boundaries between the lesion areas and the non-lesion areas may be more clearly expressed in the result image using the functions as illustrated in FIGS. 10B and 10D, in comparison to the functions as illustrated in FIGS. 10A and 10C, since voxels located in the boundaries between the lesion areas and the non-lesion areas have higher gradient values.

The 2D opacity transfer function may have a linear structure as illustrated in FIGS. 10A and 10B or a nonlinear structure as illustrated in FIGS. 10C and 10D.

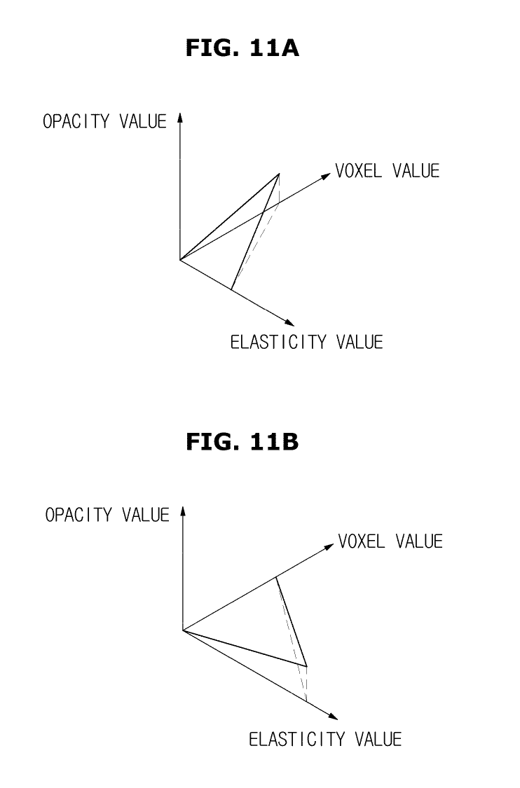

FIGS. 11A and 11B illustrate graphs of 2D opacity transfer functions using elasticity values according to an exemplary embodiment.

When the operator wants to obtain information regarding the surface of the target region rather than the inside of the target region, the opacity value may be adjusted by increasing the weight of the voxel value. That is, although the opacity value is established as a 2D function with respect to the elasticity value and the voxel value, the opacity value only increases proportionally to the voxel value. Accordingly, when the elasticity value is 0, the opacity value may be adjusted so as to be established as a 1D increasing function with respect to the voxel value.

FIG. 11A exemplarily illustrates a 2D opacity transfer function in which the weight of the voxel value is increased. By setting the elasticity value to 0, the opacity value is established as a 1D increasing function with respect to the voxel value. Thus, by setting the elasticity value to 0, the operator may obtain information regarding the surface of the target region.

When the operator wants to obtain information regarding the inside of the target region rather than the surface of the target region, the opacity value may be adjusted by increasing the weight of the elasticity value. That is, although the opacity value is established as a 2D function with respect to the elasticity value and the voxel value, the opacity value only increases proportionally to the elasticity value. Thus, when the voxel value is 0, the opacity value may be adjusted so as to be established as a 1D increasing function with respect to the elasticity value.

FIG. 11B exemplarily illustrates a 2D opacity transfer function in which the weight of the elasticity value is increased. By setting the voxel value to 0, the opacity value is established as a 1D increasing function with respect to the elasticity value. Thus, by setting the voxel value to 0, the operator may obtain information regarding the inside of the target region.

A command as to whether information to be obtained by the operator is information regarding the surface of the target region or information regarding the inside of the target region may be input using the input unit 400. The parameter adjuster 334 may set the weights of the voxel value and the elasticity value in accordance with the input command to adjust the opacity value.

The voxel value is established as a 1D increasing function with respect to the elasticity value so as to increase proportionally to the elasticity value. Here, the 1D increasing function may have a linear or nonlinear structure.

Furthermore, the voxel value may be adjusted by Equation 1 below.

Voxelₒᵤₜ=Voxelᵢₙ×ƒ(e) Equation 1

In Equation 1, e is an elasticity value and f is a value from 0 to 1. The function of the voxel value is a 1D increasing function dependent upon the elasticity value, Voxelᵢₙ is a voxel value before adjustment, and Voxelₒᵤₜ is a voxel value after adjustment. In this regard, f may be defined as a voxel value after adjustment.

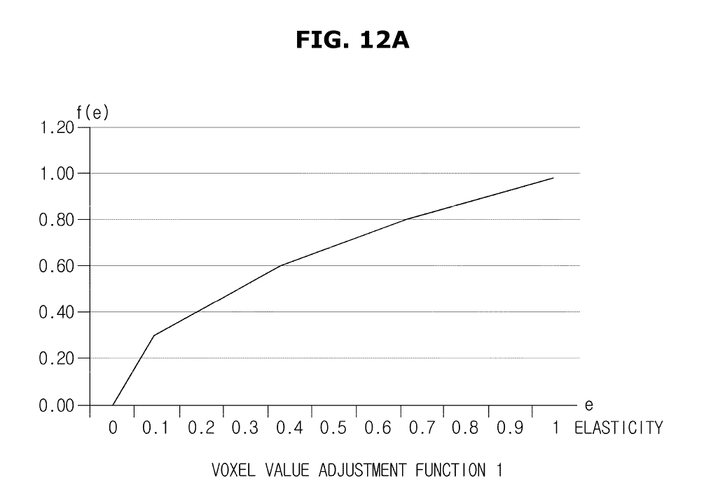

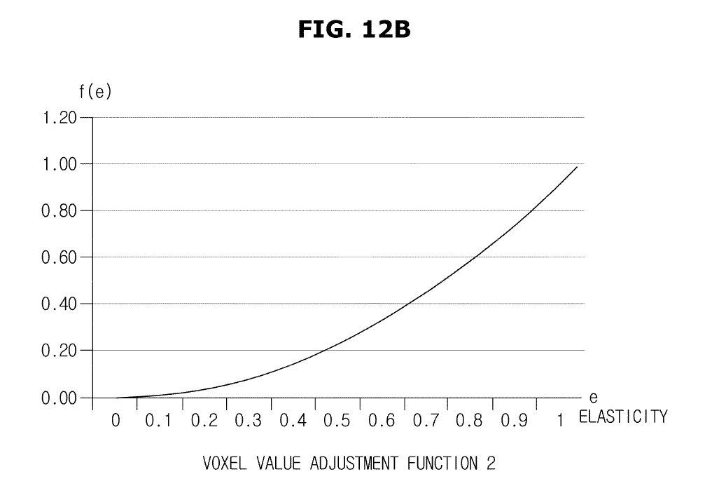

FIGS. 12A and 12B illustrate graphs of voxel value adjustment functions ƒ according to an exemplary embodiment.

Referring to FIGS. 12A and 12B, the voxel value adjustment function ƒ is a 1D increasing function proportional to the elasticity value e of 0 to 1. The voxel value also has a value of 0 to 1. Here, the graph of the voxel value adjustment function ƒ may be a 1D increasing downwardly concave function in which the slope gradually decreases as the elasticity value increases as illustrated in FIG. 12A or may be a 1D increasing upwardly concave function in which the slope gradually increases as the elasticity value increases as illustrated in FIG. 12B.

The voxel value adjustment function ƒ may have a nonlinear structure as illustrated in FIGS. 12A and 12B, but may also have a linear structure.

A command as to whether the parameter to be adjusted using the elasticity value is the opacity value or the voxel value may be input by the operator via the input unit 400 or may preset regardless of the operator's input. When the operator inputs a command to control the voxel value or controlling of the voxel value is preset, the parameter adjuster 334 adjusts the voxel value as described above, and then adjusts the opacity value of the corresponding voxel using the voxel value after adjustment. In this regard, the opacity value may be adjusted using the voxel value according to any method well known in the art.

As described above, the parameter adjuster 334 may adjust at least one of the opacity value of the voxel and the voxel value.

Then, the parameter adjuster 334 may adjust the color value of the corresponding voxel using the opacity value of the voxel and the voxel value. Here, the used opacity value and voxel value may be adjusted values as described above. The color value may be adjusted using the opacity value and the voxel value according to any one method well known in the art.

The storage 340 may store data or algorithms to manipulate the ultrasonic imaging apparatus.

The storage 340 may store a plurality of volume data generated by the volume data generator 310 and elasticity data generated by the elasticity data generator 320. That is, spatial coordinate values of the voxels, and voxel values and elasticity values corresponding thereto may be stored.

The storage 340 may also store the voxel values, the opacity values, and the color value, before and after adjustment by the parameter adjuster 334.

The storage 340 may also store image data of a resultant image generated by the image processor 350, which will be described later.

For example, the storage 340 may store algorithms such as an algorithm to generate volume data based on a plurality of 2D cross-sectional images, an algorithm to generate elasticity data based on displacement of the plurality of volume data, an algorithm to align the geometrical positions of the pluralities of volume data and elasticity data in a one-to-one corresponding manner, an algorithm to adjust the opacity value or the voxel value, an algorithm to adjust the color value, and an algorithm to perform volume rendering based on the volume data.

The storage 340 may be implemented as a storage device including a non-volatile memory device such as a read only memory (ROM), a programmable read only memory (PROM), an erasable programmable read only memory (EPROM), and a flash memory, a volatile memory such as a random access memory (RAM), a hard disk, or an optical disc. However, the disclosure is not limited thereto, and any other storages well known in the art may also be used.

The image processor 350 may include a renderer 351 and an image corrector 352.

The renderer 351 may perform volume rendering based on 3D volume data adjusted by the parameter adjuster 334 and generate a projection image of the object. Particularly, the volume rendering is performed based on the voxel values, the opacity values, and the color values constituting the volume data generated before external stress is applied to the object. If there is an adjusted value by the parameter adjuster 334, volume rendering is performed using the adjusted value.

A method of performing volume rendering by the renderer 351 is not limited. For example, ray casting may be used.

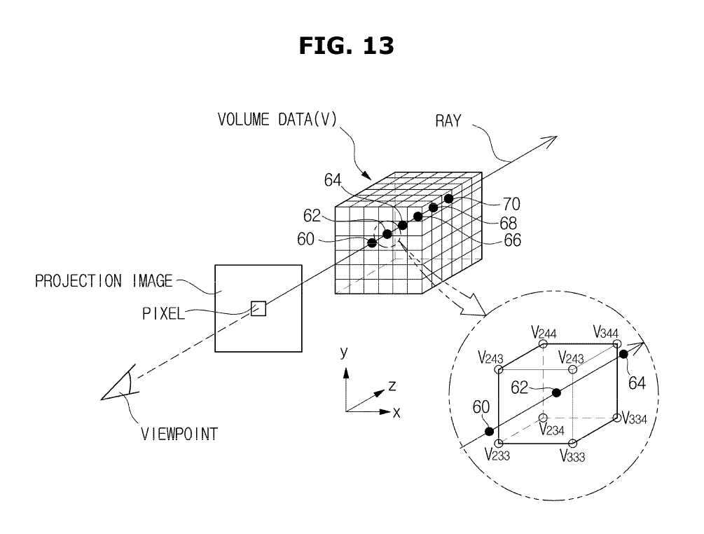

FIG. 13 is a diagram for describing volume ray casting.

Referring to FIG. 13, when an operator gazes in a direction, a straight line is formed from a viewpoint of the operator in the gazing direction. A virtual ray is emitted in the gazing direction from a pixel of an image located on the straight line. Sample points 60, 62, 64, 66, 68, and 70 are selected from 3D volume data V located on a path of the ray.

When the sample points are selected, color values and opacity values of the sample points are respectively calculated. In this regard, the color value and the opacity value of each of the sample points may be calculated via an interpolation method using color values and opacity values of voxels adjacent to each of the sample points. For example, the color value and the opacity value of sample point 62 may be calculated via interpolation of color values and opacity values of 8 voxels V₂₃₃, V₂₃₄, V₂₄₃, V₂₄₄, V₃₃₃, V₃₃₄, V₃₄₃, and V₃₄₄ adjacent to sample point 62.

The calculated color values and opacity values of the sample points are accumulated to determine the color value and the opacity value of the pixel from which the ray is emitted. In addition, an average or weighted average of the color values and the opacity values of each of the sample points may be determined as the color value or the opacity value of the pixel. Here, the determined color value and opacity value are regarded as pixel values of the pixel from which the ray is emitted.

A projection image may be generated by filling all pixels of the image by repeating the aforementioned process.

The image corrector 352 may correct brightness, contrast, color, size, or direction of the projection image generated by the renderer 351.

The image corrector 352 may transmit the corrected image to the display 500 via a wired or wireless communication network. Accordingly, the operator may confirm the corrected image of the object.

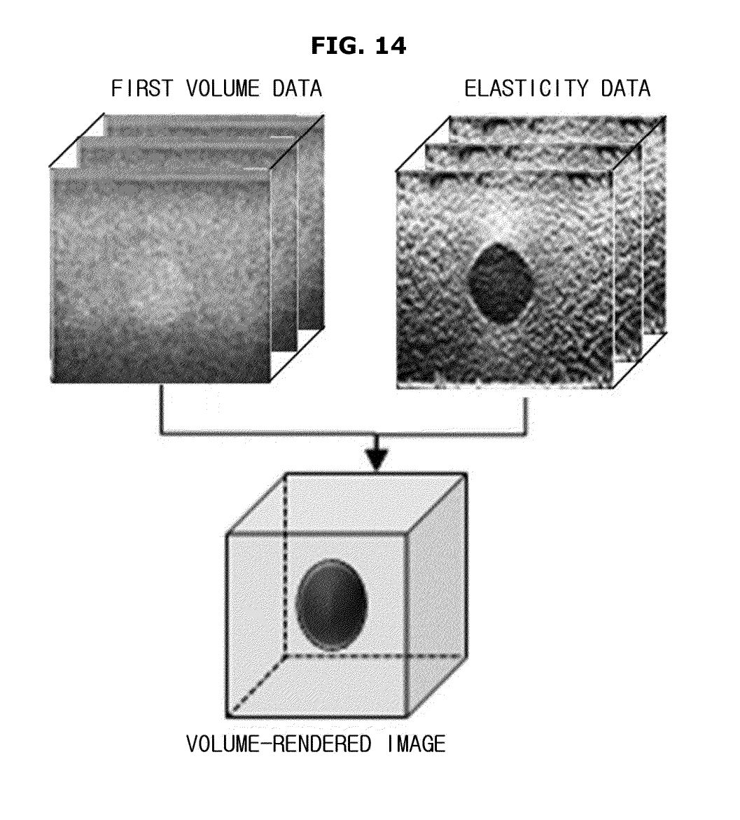

FIG. 14 is a diagram illustrating a 3D ultrasonic image acquisition by the ultrasonic imaging apparatus.

When the volume data generated before external stress is applied to the object among the plurality of volume data generated by the volume data generator 310 is first volume data, information regarding the inside of the target region, i.e., boundaries of the lesion area, may not be clearly represented by the first volume data as illustrated in FIG. 14. On the other hand, the boundaries of a lesion area having a high elasticity value may be clearly represented by the elasticity data generated based on displacement of the plurality of volume data.

Thus, when the voxel value, the opacity value, and the color value of the first volume data are adjusted using the elasticity data, and volume rendering is performed using the adjusted values, a result image in which the lesion area is clearly distinguished from non-lesion areas may be acquired as illustrated in FIG. 14

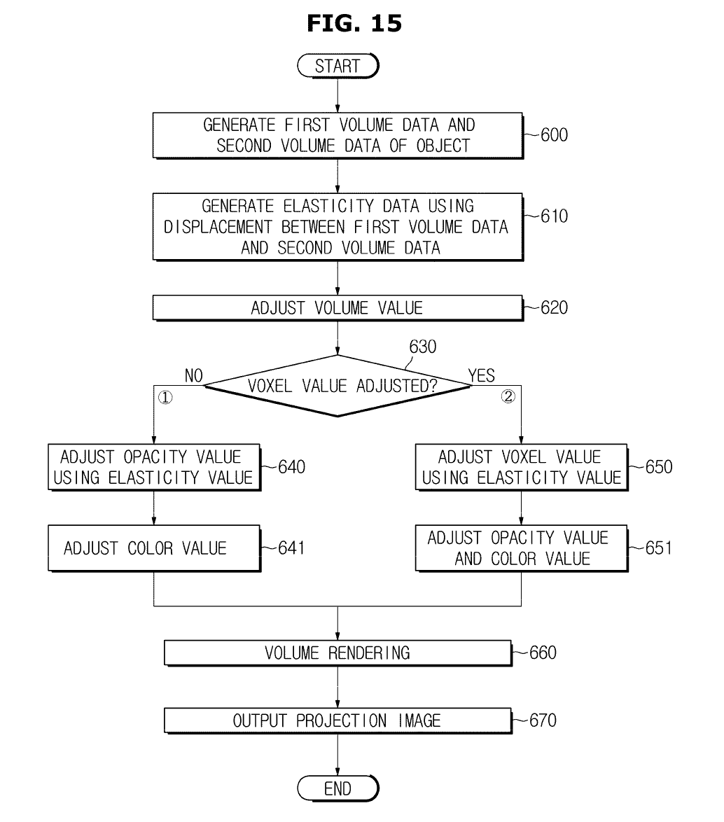

FIG. 15 is a flowchart illustrating a method of controlling an ultrasonic imaging apparatus according to an exemplary embodiment.

Referring to FIG. 15, first, the volume data generator 310 generates first volume data V and second volume data W of the object (operation 600).

Here, when the echo signal received from the object as the probe 100 transmits the ultrasonic signal before external stress is applied to the object is regarded as a first echo signal, and the echo signal received from the object as the probe 100 transmits the ultrasonic signal while external stress is applied to the object is regarded as a second echo signal, the first volume data is a set of 3D alignments corresponding to the first echo signals, and the second volume data is a set of 3D alignments corresponding to the second echo signals.

When a plurality of volume data is generated, the elasticity data generator 320 generates elasticity data E based on displacement between the first volume data V and the second volume data W (operation 610).

Before generating the elasticity data, geometrical positions of the first volume data V are aligned to the geometrical positions of the second volume data W such that voxels V_{xyz} of the first volume data V respectively correspond to voxels W_{xyz} of the second volume data W.

Displacements between voxels of the corresponding first volume data and second volume data are respectively calculated, and then elasticity values of the voxels are respectively calculated based on the displacements.

Here, when the target region contains cancerous or tumor-like lesions, the voxel values of the lesions are not significantly changed by external stress, such that the calculated elasticity values increase. On the other hand, in soft tissues which are non-lesion areas, displacement of the voxels increases by the external stress, such that the calculated elasticity values decrease.

The elasticity data form a set of 3D alignments, similarly to first and second volume data V and W. Here, the voxel values of the voxels constituting the elasticity data indicate elasticity values.

Then, volume is adjusted between the first volume data V and the elasticity data E (operation 620). That is, the geometrical positions of the first volume data V are adjusted to geometrical positions of the elasticity data E such that the voxels V_{xyz} of the first volume data V respectively correspond to voxels E_{xyz} of the elasticity data E.

When the volume is adjusted, necessity of adjusting the volume value is determined by the operator or a preset method (operation 630).

If a command not to adjust the voxel value is input by the operator or preset (\textcircled{1}), the parameter adjuster 334 adjusts the opacity value using the elastic value of the elasticity data E (operation 640).

The opacity value is established as a 1D increasing function with respect to the elasticity value so as to increase proportionally to the elasticity value.

The opacity value may be established as a 2D increasing function with respect to the elasticity value and the voxel value so as to increase proportionally to the elasticity value and the voxel value. Alternatively, the opacity value may be established as a 2D increasing function with respect to the elasticity value and the gradient value so as to increase proportionally to the elasticity value and the gradient value.

When the operator wants to obtain information regarding the surface of the target region rather than the inside of the target region, the opacity value may be established as a 2D function with respect to the elasticity value and the voxel value, while only increasing proportionally to the voxel value. Thus, when the elasticity value is 0, the opacity value may be established as a 1D increasing function with respect to the voxel value.

When the operator wants to obtain information regarding the inside of the target region rather than the surface of the target region, the opacity value may be established as a 2D function with respect to the elasticity value and the voxel value, while only increasing proportionally to the elasticity value. Thus, when the voxel value is 0, the opacity value may be established as a 1D increasing function with respect to the elasticity value.

In this regard, the operator may input whether the information to be acquired is information regarding the surface of the target region or information regarding the inside of the target region.

The parameter adjuster 334 adjusts a color value of a corresponding voxel using the opacity value and the voxel value (operation 641).

In this regard, the opacity value after adjustment is used, the method of adjusting the color value using the opacity value and the voxel value may be any known method in the art, and thus a detailed description thereof will not be given.

If a command to adjust the voxel value is input by the operator or is preset \textcircled{2}, the parameter adjuster 334 adjusts the voxel value using the elasticity value of the elasticity data E (operation 650).

The voxel value is established as a 1D increasing function with respect to the elasticity value and may be adjusted so as to increase proportionally to the elasticity value.

The voxel value may be adjusted by Equation 1 below.

Voxelₒᵤₜ=Voxelᵢₙ×ƒ(e) Equation 1

In Equation 1, e is an elasticity value, f is a value from 0 to 1. The function of the voxel value is a 1D increasing function dependent upon the elasticity value, Voxelᵢₙ is a voxel value before adjustment, and Voxelₒᵤₜ is a voxel value after adjustment.

The parameter adjuster 334 adjusts the opacity value of the corresponding voxel using the voxel value, and then adjusts the color value of the corresponding voxel using the opacity value and the voxel value (operation 651).

Here, the voxel value and opacity value after adjustment are used, a method of adjusting the opacity value using the voxel value or a method of adjusting the color value using the opacity value and the voxel value are well known in the art, and thus a detailed description thereof will not be given.

The parameters adjusted as described above may include the voxel value, the opacity value, and the color value of each voxel constituting the first volume data V.

When the parameters of volume rendering are adjusted, volume rendering is performed based on the adjusted first volume data V (operation 660).

That is, the volume rendering is performed by using the voxel value, the opacity value, and the color value of the voxels constituting the first volume data V and adjusted by the parameter adjuster 334.

The method of performing volume rendering is not limited. For example, volume ray casting may be used. Volume ray-casting may be performed by selecting sample points from the first volume data V corresponding to each pixel of an image, calculating a color value and a transparency value of each of the sample points via interpolation of adjacent voxels, and calculating a color value and a transparency value of each pixel by accumulating the calculated color values and transparency values.

A projection image of the object may be generated by performing volume rendering, and brightness, contrast, or color of the projection image may further be corrected.

The generated projection image may be output to the display 500 connected to the main body 300 via a wired or wireless communication network (operation 670).

Accordingly, the operator may confirm the result image of the object displayed on the display screen implemented as a cathode ray tube (CRT), a liquid crystal display (LCD), an organic light emitting diode (OLED) display, and the like.

As apparent from the above description, according to the ultrasonic imaging apparatus and the control method thereof, a multi-dimensional ultrasonic image of a target region in which lesion areas and non-lesion areas are separated from each other may be output. Thus, both the information regarding the surface of the target region and the information regarding the inside, i.e., internal volume, of the target region may be acquired.

The described-above exemplary embodiments and advantages are merely exemplary and are not to be construed as limiting. The present teaching can be readily applied to other types of apparatuses. The description of exemplary embodiments is intended to be illustrative, and not to limit the scope of the claims, and many alternatives, modifications, and variations will be apparent to those skilled in the art.

What is claimed is:

1. An ultrasonic imaging apparatus comprising:
an ultrasonic probe;
a volume data generator configured to generate a plurality of volume data corresponding to echo signals received as the ultrasonic probe transmits the ultrasonic signals to an object a plurality of times before and while external stress is applied to the object;
an elasticity data generator configured to generate elasticity data based on displacement of the plurality of volume data;
a controller configured to adjust parameters of volume rendering using the elasticity data; and
an image processor configured to perform the volume rendering using the adjusted parameters and generate a volume-rendered image.

2. The ultrasonic imaging apparatus according to claim 1, wherein the volume data generator generates first volume data corresponding to first echo signals received as the ultrasonic probe transmits the ultrasonic signals to the object before the external stress is applied to the object, and generates second volume data corresponding to second echo signals received as the ultrasonic probe transmits the ultrasonic signals to the object while the external stress is applied to the object.

3. The ultrasonic imaging apparatus according to claim 1, wherein the parameters adjusted by the controller comprise at least one of an opacity value of a voxel and a voxel value.

4. The ultrasonic imaging apparatus according to claim 3, wherein the opacity value is established as a one-dimensional increasing function with respect to the elasticity value and is adjusted proportionally to the elasticity value.

5. The ultrasonic imaging apparatus according to claim 3, wherein the opacity value is established as a two-dimensional (2D) increasing function with respect to the elasticity value and the voxel value and is adjusted proportionally to the elasticity value and the voxel value, or
the opacity value is established as the 2D increasing function with respect to the elasticity value and a gradient value and is adjusted proportionally to the elasticity value and the gradient value.

6. The ultrasonic imaging apparatus according to claim 3, wherein the opacity value is established as a two-dimensional increasing function with respect to the elasticity value and the voxel value while only increasing proportionally to the voxel value and is adjusted to be established as a one-dimensional increasing function with respect to the voxel value when the elasticity value is 0.

7. The ultrasonic imaging apparatus according to claim 3, wherein the opacity value is established as a two-dimensional increasing function with respect to the elasticity value and the voxel value while only increasing proportionally to the voxel value and is adjusted to be established as a one-dimensional increasing function with respect to the elasticity value when the voxel value is 0.

8. The ultrasonic imaging apparatus according to claim 3, wherein the voxel value is established as a one-dimensional increasing function with respect to the elasticity value and is adjusted proportionally to the elasticity value.

9. The ultrasonic imaging apparatus according to claim 3, wherein a function of the voxel value is a one-dimensional increasing function dependent upon an elasticity value, and
the voxel value is adjusted as:
Voxelₒᵤₜ=Voxelᵢₙ×ƒ(e),
wherein e is the elasticity value,
f is a value from 0 to 1,
Voxelᵢₙ is a voxel value before adjustment, and
Voxelₒᵤₜ is a voxel value after the adjustment.

10. The ultrasonic imaging apparatus according to claim 3, wherein the parameters adjusted by the controller further comprise a color value of the voxel.

11. The ultrasonic imaging apparatus according to claim 1, further comprising a volume data adjuster configured to align geometrical positions of the plurality of volume data and geometrical positions of the elasticity data.

12. A method of controlling an ultrasonic imaging apparatus, the method comprising:
receiving echo signals as a probe transmits ultrasonic signals to an object a plurality of times before and while external stress is applied to the object;
generating a plurality of volume data corresponding to the echo signals;
generating elasticity data based on displacement of the plurality of volume data;
adjusting parameters of volume rendering using the elasticity data;
performing volume rendering using the adjusted parameters; and
generating a volume rendered image.

13. The method according to claim 12, wherein the adjusting the parameters of volume rendering comprises adjusting at least one of an opacity value of a voxel and a voxel value.

14. The method according to claim 13, wherein the opacity value is established as a one-dimensional increasing function with respect to the elasticity value and is adjusted proportionally to the elasticity value.

15. The method according to claim 13, wherein the opacity value is established as a two-dimensional (2D) increasing function with respect to the elasticity value and the voxel value and is adjusted proportionally to the elasticity value and the voxel value, or
the opacity value is established as the 2D increasing function with respect to the elasticity value and a gradient value and is adjusted proportionally to the elasticity value and the gradient value.

16. The method according to claim 13, wherein the opacity value is established as a two-dimensional increasing function with respect to the elasticity value and the voxel value while only increasing proportionally to the voxel value and is adjusted to be established as a one-dimensional increasing function with respect to the voxel value when the elasticity value is 0.

17. The method according to claim 13, wherein the opacity value is established as a two-dimensional increasing function with respect to the elasticity value and the voxel value while only increasing proportionally to the voxel value and is adjusted to be established as a one-dimensional increasing function with respect to the elasticity value when the voxel value is 0.

18. The method according to claim 13, wherein the voxel value is established as a one-dimensional increasing function with respect to the elasticity value and is adjusted proportionally to the elasticity value.

19. The method according to claim 13, wherein a function of the voxel value is a one-dimensional increasing function dependent upon an elasticity value, and
the voxel value is adjusted as:
Voxelₒᵤₜ=Voxelᵢₙ×ƒ(e),
wherein e is the elasticity value,
f is a value from 0 to 1,
Voxelᵢₙ is a voxel value before adjustment, and
Voxelₒᵤₜ is a voxel value after the adjustment.

20. The method according to claim 13, wherein the adjusting the parameters of volume rendering further comprises adjusting a color value of the voxel.
